(19) United States
(12) Patent Application Publication
(10) Pub. No.: US 2017/0049628 A1
(43) Pub. Date: Feb. 23, 2017

(54) Snap Compression Bandage and Wrap

(71) Applicant: Michael J. Stevens, Houston, TX (US)

(72) Inventor: Michael J. Stevens, Houston, TX (US)

(21) Appl. No.: 15/069,876

(22) Filed: Mar. 14, 2016

Related U.S. Application Data
(63) Continuation of application No. 13/815,496, filed on Mar. 7, 2013, now abandoned.

(51) Int. Cl. A61F 13/00; A61M 35/00

(52) U.S. Cl. CPC A61F 13/00085; A61F 13/00029; A61F 13/00038; A61F 13/00063; A61M 35/00; A61F 2013/00106; A61F 2013/0028

(57) ABSTRACT

The present invention relates to a quick application bandage for medical applications to prevent instant loss of blood or other bodily fluid. The bandage does not require any adhesive, straps or other attachment applications for quick attachment to the living appendage. The bandage is constructed of deformable metal members or bands embedded in padded bandage materials to enable compression around the injured area or bodily member. The snap compression bandage is adaptable for reuse and will return to its original shape after each application. The bandage is especially useful for first responders or military personnel such in naval or army operations where injuries may require immediate urgent attention.

### BACKGROUND OF THE INVENTION AND PRIOR ART

The invention relates generally to a special bandage application which applies easily to injuries without the inconvenience of adhesives and or other methods of attachment to the injured limb or member of the human body or an animal body.

The limitations of the prior art in this area suggests the need for such an improved bandage for ease of use and ease of application to an injured part of the human body or to an animal body for therapeutic and medical treatment remedy.

U.S. Pat. No. 7,264,630 issued Sep. 4, 2007, to Webb teaches a hot/cold therapy pack which is attached to the human body by rigid member which is supported by a skeletal structure. The skeletal structure has a plurality of elongated leg portions that wrap around the human body part for treatment. The particular application of this bandage does not indicate that the supporting skeletal structure may be repeatedly used with no deformation from its original sizing.

U.S. Pat. No. 7,837,638 issued Nov. 23, 2010, to Miros teaches a heat exchange bladder with flexible attachment devices which is attached to the afflicted or injured body area by means of a tied attachment. The bladder has an opening at a center section that provides flexibility. The cover of the bladder may comprise a nylon loop outer surface to allow the upper lower sections to be easily attached and adjusted. The invention requires an exchange bladder with an opening and does not easily conform to the size of the body part to be treated, and instead requires straps to tie on the bladder.

U.S. Pat. No. 5,409,500 issued Apr. 25, 1995, to Dyrek describes a therapy pack using Velcro fasteners. The Dyrek teaching recites Velcro as a means of fastening the therapy to the patient. The patent describes elastomeric straps having restricted micro-fastener areas for versatile connection on the anatomical surface. The structure requires Velcro which will wear over time and thus not be useful to attach the therapy pack to an injured body part.

U.S. Pat. No. 6,440,159 issued Aug. 27, 2002, to Edwards et al., also describes a therapy neoprene wrap using Velcro fasteners. The disadvantage of Velcro fasteners being that they do not snap into position and under continual wear will no longer successfully fasten or secure the bandage wrap.

U.S. Pat. No. 5,165,402 issued Nov. 24, 1992, to McCoy also describes a therapy wrap using Velcro fasteners. The disadvantage of Velcro fasteners being that they do not snap into position and under continual wear will no longer successfully fasten or secure the bandage.

U.S. Pat. No. 5,111,810 issued May 12, 1992, to Fortney teaches a therapy wrap using buckle fasteners. The wrap is applied with a thermal pack using straps and buckle fasteners around the affected appendage. The disadvantage of buckle fasteners is the difficulty of fastening the buckles on a large wound or body part injury. The additional disadvantage is that the buckles may break or wear so that they no longer function for securing the therapy wrap.

In view of these teachings and disclosures of bandages or medical wraps for attaching bandages to injured areas or limbs of the human body, it is apparent that that there is a need for a more convenient device or apparatus to apply bandages to an injury. This device or apparatus would overcome the disadvantages of the prior art by being reusable, readily conformable to the injured area, and quick to apply in any environmental context.

### SUMMARY OF THE INVENTION

The present invention relates to a quick application bandage for medical applications to prevent instant loss of blood or other bodily fluid and for treatment of bodily injuries by the instant application of medicine through the bandage. The invention of a snap compression bandage may be used to apply a bandage or therapeutic wrap to a human body injury or to an animal injury.

An advantage of the invention is that the bandage does not require any adhesive, straps or other attachment applications for quick attachment to the injured bodily limb or appendage. The bandage is constructed of a layer of deformable metal members embedded in padded materials to enable compression around the injured area or body member. On top of the layer of deformable metal members is a cloth layer which may be applied to the injured area.

Another advantage of the invention is that the snap compression bandage is adaptable for reuse and will return to its original shape after each application.

Another advantage of the invention is that the metal members that form the underlying structure of the bandage deform and curl under pressure to take the shape of the appendage being bandaged.

Another advantage of the invention is that when the injury is examined or cleaned, the snap compression bandage may be removed as the metal members will deform to their original size and dimension for reapplication to the injured body area.

Another advantage of the invention is that the snap compression bandage may hold a bandage, a hot/cold gel pack, a bandage laced with medicine including pain relievers such as “Novocain,” or a bandage laced with antibiotics in a pre-packaged delivery system for immediate application to the injured body limb or member.

Another advantage of the invention is that the snap compression bandage may be made in variable sizes to accommodate the injury limbs of a large person or large animal or small person or small animal.

Another advantage of the invention is that the snap compression bandage, in view of its quick application and versatile construction, may be used in a number of adverse environments such as desert conditions, in extreme hot environments with temperatures over 100 degrees Fahrenheit or extreme cold environments with temperatures under 32 degrees Fahrenheit, as well as underwater environments.

Another advantage of the invention is that it may be used in urgent care applications for first responders, police, or military. With regard to military, it may be used by Navy Seal Team personnel or by Army Ranger personnel when immediate application of a pressurized bandage may be needed to prevent loss of blood or even to maintain the viable integrity of an injured appendage.

### BRIEF DESCRIPTION OF THE DRAWINGS

FIG. 1 is an illustration of a human hand and arm with the attached snap compression bandage wrapped around an injured portion of the arm and hand.

FIG. 2 is a drawing of the snap compression bandage, open and ready to be applied.

FIG. 3 is a drawing of the snap compression bandage closed, as when applied to an injured limb.

FIG. 4 is a drawing of the side cross section of the snap compression bandage, showing the compressing bands of the bandage covered by a protective covering.

FIG. 5 is a drawing of the snap compression bandage with the extremities of the bandage rolled up and under compression.

FIG. 6 is a thawing of the snap compression bandage being applied to a human wrist and arm, with one side compressing around the human arm and the other side about to be applied to the human arm.

FIG. 7 is a flow chart showing the method of using and applying the snap compression bandage.

### DETAILED DESCRIPTION OF THE INVENTION

The present invention relates to a quick application bandage for medical applications to prevent instant loss of blood or other bodily fluid and for treatment of bodily injuries by the instant application of medicine through the bandage.

The features of the invention are presented in the figures and elements of the figures to illustrate the construction and application of the invention

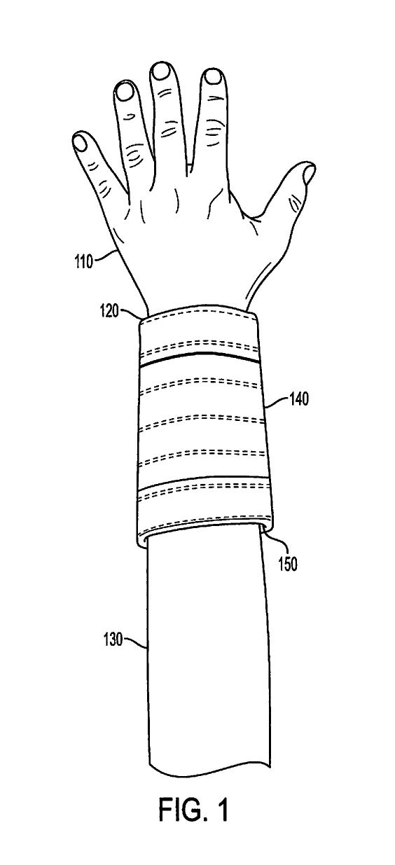

A demonstration of the essential application of the invention snap compression bandage is shown in FIG. 1; the human hand 110, the human wrist 120, and the human arm 130 have been injured and the snap compression bandage 140 has been applied to the affected area. The snap compression bandage 140 has been wrapped around the affected area to provide protection from environmental contamination as well as medication 150 to the affected area. The bandage is shown around the human wrist and hand but may be designed to cover various size limbs and variable size injuries or various sizes of afflicted areas.

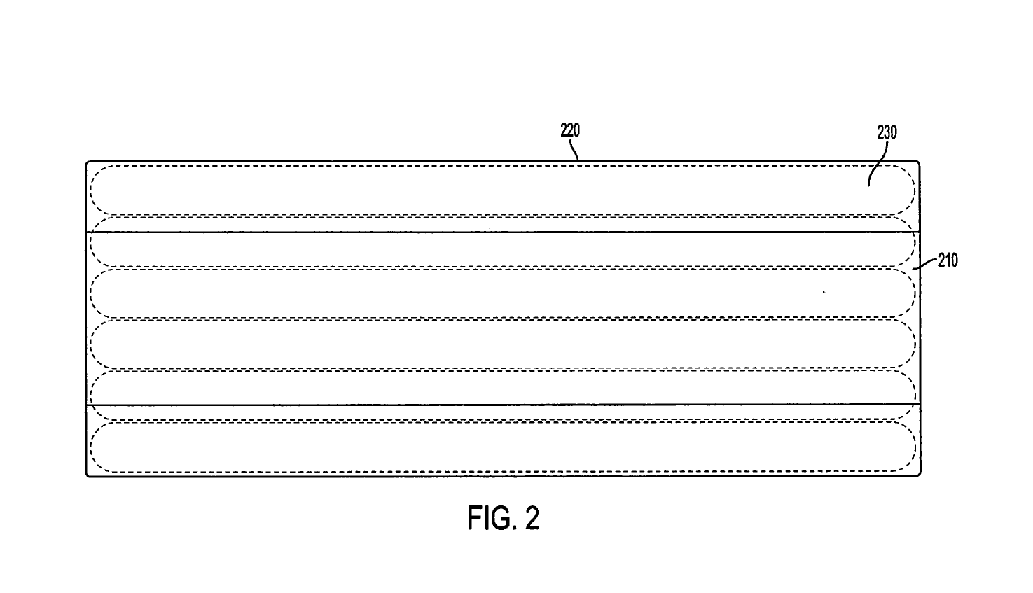

A demonstration of the construction of the invention snap compression bandage is shown in FIG. 2. The invention is constructed from two layers of material. One layer is cloth and is usually sterile woven cotton cloth 210 and is located entirely on one side which will face and adhere to the injured body limb. The other layer or backing of the snap compression bandage 220 is constructed from a layer of flexible metal bands 230 which bend around the injured body limb. The metal bands 230 are made of flexible metal of the type normally used for a carpenter's flexible tape measure and are bendable metal strips of uniform length. The metal bands are bistable spring metal, which will coil when slapped against the appropriate object such as a human wrist for example. The slap on band exists in two states; the elongated state, and the coiled state. These metal bands may be stretched to lie flat or to curl around the affected body limb. The bandage with the layer of flexible metal bands deforms around the injured bodily member or limb.

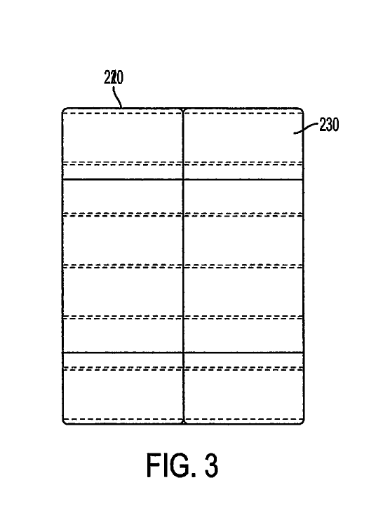

FIG. 3 shows a top view projection of the contracted snap compression bandage when the metal bands 230 have contracted around a body limb (not shown). The backing of the contracted bandage 210 contains the bandage material which is laced with anti-pain or anti-bacterial or other medicinal or therapeutic treatment that is needed to be transferred to the injured body tissue.

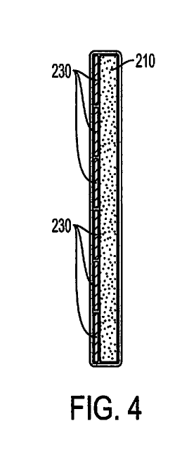

Further, FIG. 4 illustrates the side view of the invention snap compression bandage showing the layer of cloth bandage material 210 on the top which will face the injured body limb. The side view of FIG. 4 also shows the layer 230 below the bandage material consisting of the metal bands which will contract around the injured body limb. The cloth bandage material 210 transmits medicine in the form of anti-pain or anti-bacterial treatment or other therapeutic treatment to the affected area. A preferred cloth layer would be made of sterilized woven cotton. The snap compression bandage can be pretreated with a permeable membrane with directional usage to automatically drain bodily fluids away from the damaged appendage/body part to speed healing of a wound. The cloth layer of the snap compression bandage may also be fitted with a fiber fill as an additional absorbent layer.

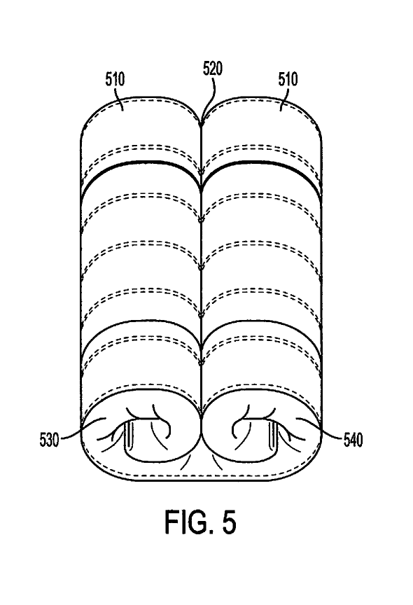

The invention snap compression bandage, as it would be packaged and delivered to consumers, is shown in FIG. 5. Each half of the bandage 510 is rolled to the center 520 so that the bandage will occupy minimal space for shipping, storage, and transport to medical support applications. FIG. 5 shows the bandage material 530 before application with therapeutic treatments for transfer to the injured body limb. FIG. 5 also shows the curl of the contracting bandage members 540 which will expand when the bandage is applied to the injured body limb or wound.

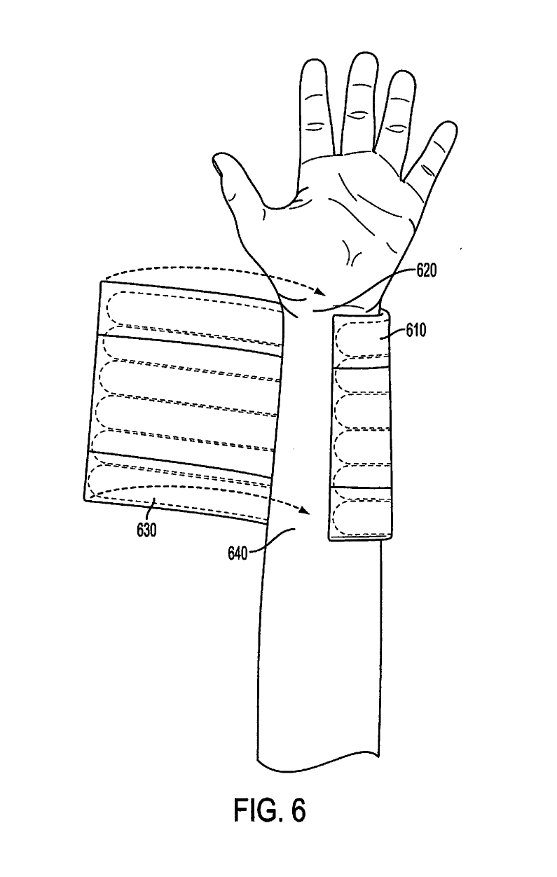

The application of the invention snap compression bandage to an injured limb is shown in FIG. 6. The injured limb or arm is positioned for insertion into the bandage which compresses around the injured limb, one half of the bandage at a time. One half of the bandage 610 is compressed around a human patient's injured wrist 620. The other half of the bandage 630 is then allowed to close on the remainder of the exposed wrist 640. The opening and closing of the bandage around the injured bodily member may be done with repeated applications and with repeated treatments of anti-pain medications or anti-bacterial medication or other various therapeutic medications. The application of therapeutic medications using this quick compression bandage has advantages for the emergency responders or war fighting personnel for treatment of human injuries. The bandage may also be used in a veterinary context for treatment of animal injuries.

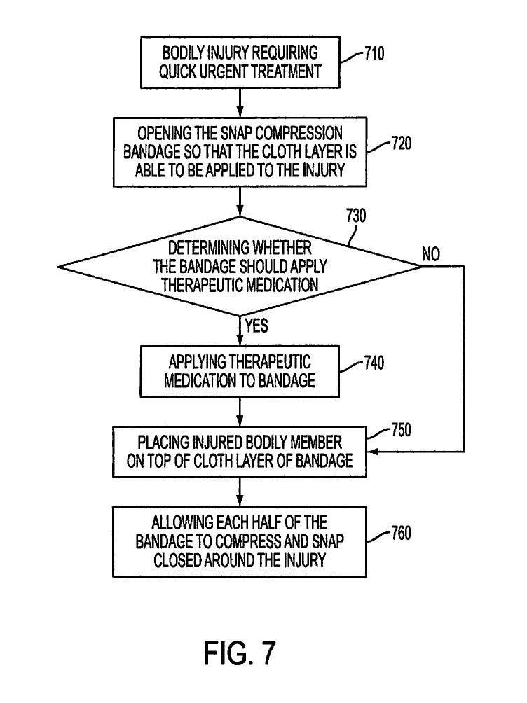

The method of using the snap compression bandage is shown by the steps in FIG. 7. The first step 710 is determining that there is an injury that requires quick and urgent treatment. The second step 720 is opening the bandage containing a cloth layer with a metal band layer below the cloth layer so that the metal band layer is flat and the cloth layer above the metal band layer is able to be applied to the injured bodily member or limb. The third step 730 is determining whether the bandage should have therapeutic medication applied to the cloth layer. The fourth step 740 is applying the medication if needed. The fifth step 750 is placing the injured bodily member on top of the cloth layer of the bandage. The sixth step 760 is allowing each half of the bandage to compress and snap closed around the injured limb.

The invention of a snap compression bandage, in view of its quick application and versatile construction, may be used in a number of adverse environments such as desert conditions, in extreme hot environments with temperatures over 100 degrees Fahrenheit or extreme cold environments with temperatures under 32 degrees Fahrenheit, as well as underwater environments.

The invention is capable of being used in urgent care applications for first responders, police, or military. With regard to military, it may be used by Navy Seal Team personnel or by Army Ranger personnel when immediate application of a pressurized bandage may be needed to prevent loss of blood or even to maintain the viable integrity of an injured appendage. The quick application of the snap compression bandage permits application with a few seconds of opening the bandage and compressing it on the wound or injured limb or appendage.

When introducing elements of the present invention or the preferred embodiment(s) thereof, the articles “a,” “an,” “the,” and “said” are intended to mean there are one or more of the elements. The terms “comprising,” “including,” and “having” are intended to be inclusive and mean that there may be additional elements other than the listed elements.

Although the present invention has been described in considerable detail with reference to a certain preferred embodiments thereof, other embodiments are possible. Therefore, the spirit and scope of the appended claims should not be limited to the description of the preferred embodiment(s) contained herein.

1-13. (canceled)

14. (canceled)

15. A bandage for urgent care and medical applications, comprising:
a cloth layer for application to an injured limb,
said cloth layer further comprising a sterile woven cotton cloth containing medications for wrapping around an injured limb for medicinal treatment of the injured limb;
a layer of flexible metal bands attached to said cloth layer for clamping said cloth layer on said injured limb,
said flexible metal bands further comprising bendable metal strips made of bistable spring metal which further deform to open the cloth layer for insertion of the injured limb and then deform to clamp closed on the injured limb.

16. The bandage of claim 15, wherein the metal bands are deformed to apply to said injured limb and then snap into place compressing around the injured limb.

17. The bandage of claim 15, wherein the metal bands are made of bendable metal strips of uniform length.

18. The bandage of claim 15, wherein the bandage with the metal band layer is deformable and formable for repeated application to injured limbs.

19. The bandage of claim 18, wherein the bandage is usable for quick application in either extreme hot or extreme cold environments.

20. The bandage of claim 18, wherein the bandage is usable under underwater.

21. The bandage of claim 18, where in the bandage is adaptable to be made in various sizes to accommodate variable size injuries.

22. The bandage of claim 15, wherein the cloth layer contains anti-pain medications.

23. The bandage of claim 15, wherein the cloth layer contains antibiotic medications.

24. The bandage of claim 15, wherein the cloth layer contains various therapeutic medications.

25. The bandage of claim 15, wherein the bandage is usable for therapeutic treatment of human or animal injuries.

26. A bandage for urgent care and medical applications, comprising:
a cloth layer for application to an injured limb,
said cloth layer further comprising a sterile woven cotton cloth for wrapping around an injured limb for medical treatment, wherein said cloth layer contains various therapeutic medications for treatment of the injured limb;
a layer of flexible metal bands attached to said cloth layer for clamping said cloth layer on said injured limb,
said flexible metal bands further comprising bendable metal strips made of bistable spring metal which further deform to open the cloth layer for insertion of the injured limb and then deform to clamp closed on the injured limb.
